(19) United States
(12) Patent Application Publication
(10) Pub. No.: US 2024/0210737 A1
(43) Pub. Date: Jun. 27, 2024

(54) FACE MASK WITH BLUE LIGHT BLOCKING LENSES

(71) Applicant: Marian Fox, Monument, CO (US)

(72) Inventor: Marian Fox, Monument, CO (US)

(21) Appl. No.: 18/599,844

(22) Filed: Mar. 8, 2024

Related U.S. Application Data
(63) Continuation-in-part of application No. 17/851,354, filed on Jun. 28, 2022.

(51) Int. Cl. G02C 7/10; A61F 9/04

(52) U.S. Cl. CPC G02C 7/104; A61F 9/045

(57) ABSTRACT

A sleep mask with blue light blocking lenses includes a soft body member having at least one lens configured to block blue light disposed therein. The mask is configured for comfort and secures over the orbital region of a wearer's face. The mask may include a padding to maintain the at least one lens away from contact with the wearer, even when a wearer lies face down wearing the mask. The at least one lens may be rendered of a flexible, non-rigid, translucent polymer. The wearer may therefore wear the mask while viewing screens in bed without exposure to blue light stimulating wakefulness.

### CROSS REFERENCE TO RELATED APPLICATIONS

This application is a continuation-in-part of U.S. patent application Ser. No. 17/851,354, filed Jun. 28, 2022, the contents of which are herein incorporated by reference.

### BACKGROUND OF THE INVENTION

### 1. Field of the Invention

Embodiments of the invention relate generally to a face mask with blue light blocking lenses that is devised to present a comfortable means of blocking blue light to prevent stimulating wakefulness when viewing screens of peripheral and electronic devices when lying down, or prior to falling asleep.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Various types of sleep masks are known in the art. All present an opaque surface wearable over the eyes of a wearer to prevent light from reaching the wearer's eyes and assist sleeping in lighted conditions. Sleep masks seen in the present state of the art do not accommodate open eyes, but are devised to be worn with the eyes closed. Such masks typically contact the closed eyelids of the wearer and cover the face above the cheeks and along the forehead of the wearer to present an opaque barrier that prevents passage of light therethrough.

Blue light blocking lenses are also known in the art. However, such lenses are typically mounted in eyewear for use during waking hours, when working and alert. Such lenses are typically mounted in rigid frames for support upon the bridge of the nose and ears in like manner of eyewear seen in the art.

What is needed is a novel combination of such art, wherein pliable blue light blocking lenses are combined into a sleep mask to enable a user to view screens and peripheral devices in bed without stimulating wakefulness due to blue light exposure. The present invention, then, enables a wearer to fall asleep while watching television, say, or to otherwise wear the mask with eyes open in viewing screens on peripheral and electronic devices before bedtime without interfering with the person's sleep cycles. Further, the present invention accommodates comfort when the wearer falls asleep wearing the mask.

### SUMMARY OF THE INVENTION

The term “blue light”, as used herein throughout, is taken to mean light of wavelengths in the range of 250 nm to 650 nm and may encompass nonvisible ultraviolet light.

Blue light is known to interfere with human sleep cycles and circadian rhythms. Due to these wavelengths of light scattering during daytime, the human species has evolved to respond to blue light stimulus by wakefulness. However, in the modern era, it has become increasingly common for people to view peripheral devices such as smart phones and tablets in bed prior to falling asleep. Peripheral devices such as these, as well as televisions and other luminous electronic devices presenting images and display, frequently emit light wavelengths across the visible spectrum, including blue light. Such activity, then, may impact a person's ability to fall asleep due to the blue light emitted from such devices. Rather than winding down, then, viewing peripheral devices in bed may excite wakefulness due to blue light stimulation.

The present sleep mask with blue light blocking lenses, therefore, has been devised to enable a comfortable means of sleeping while wearing a mask that is capable of filtering blue light from the eyes. Most blue light blocking eyewear are rigid and non-conformable to the face. Should a person fall asleep wearing such eyewear, they often are awoken by the discomfort caused when the eyewear is pressed against their face.

The present invention, then, allows a person to don the sleep mask with blue light blocking lenses, to watch television in bed, for example, or interact with a peripheral device (such as a tablet or smart phone, say) and then fall asleep without the risk of being awoken when sleeping with the mask on. The mask is comfortable and engineered to maintain the lenses removed from contact with the wearer's face. Thus, the mask may be worn with eyes open as well as with eyes closed and a wearer can fall asleep while filtering blue light from reaching the eyes.

The present sleep mask with blue light blocking lenses, therefore, includes a soft body member configured to fit comfortably over the face of a wearer. In an example embodiment contemplated herein, the soft body member is rendered from a soft and conformable fabric that is comfortable to wear and yielding when pressed against the wearer's face so that sleeping facedown with the mask worn is not uncomfortable.

Example fabrics from which the body member may be made include silk, cotton, hemp, satin, velvet, and other soft, conformable fabrics that are comfortable to wear. Additional non-fabric materials, such as soft and yielding polymers are also contemplated as within scope of this disclosure.

The present sleep mask with blue light blocking lenses further includes a pair of lenses configured to filter out blue light. The lenses may be pliable, rendered of a transparent polymer that is non-rigid and capable of filtering out certain wavelengths of light, specifically shorter wavelengths of light such as blue light. Such materials include polycarbonate and Trivex® for example, but the lenses contemplated may be more pliable and flexible than polycarbonate or Trivex®.

A soft or pliant strap member is disposed to secure the mask overtop the wearer's face. In some embodiments contemplated herein, the strap may be elasticized and disposed interior to a soft fabric sheath. In other embodiments, the strap may be nonelastic such as a ribbon or other fabric that is attachable together behind the head of the wearer. Further, the mask may include a padding centrally disposed proximal an upper edge of the body member and disposed to contact at least the bridge of the wearer's nose. The padding enables the mask to fit softly and snugly against the wearer's face, even when pressured against the wearer such as when the wearer is lying face down while wearing the mask. The padding prevents direct contact of the lenses with the wearer. Thus, the sleep mask conforms to the wearer's face as a soft surface against which the wearer is not discomforted when placing pressure against the mask when worn.

Thus has been broadly outlined the more important features of the present sleep mask with blue light blocking lenses so that the detailed description thereof that follows may be better understood and in order that the present contribution to the art may be better appreciated.

Embodiments of the present invention provide a face mask with blue light blocking lenses comprising a soft body member configured to fit comfortably over the orbital region of the face of a wearer; a padding about at least a portion of a perimetric interior surface of the soft body member; a pair of lenses configured to filter out blue light disposed in the soft body member; and a soft or pliant strap member disposed to secure the face mask upon the wearer's face, wherein the face mask conforms to the wearer's face as a soft surface against which the wearer is not discomforted when placing pressure against the face mask when worn; and the padding permits eyes of the wearer to open and close without contacting the face mask.

Embodiments of the present invention provide a sleep mask with blue light blocking lenses comprising a soft body member configured to fit comfortably over the orbital region of the face of a wearer; a pair of lenses configured to filter out blue light disposed in the soft body member; a padding about at least a portion of a perimetric interior surface of the soft body member; and a soft or pliant strap member disposed to secure the sleep mask upon the wearer's face, wherein the sleep mask conforms to the wearer's face as a soft surface against which the wearer is not discomforted when placing pressure against the sleep mask when worn; the pair of lenses consist essentially of a first lens and a second lens disposed in a non-overlapping, side-by-side arrangement; and the padding permits eyes of the wearer to open and close without contacting the sleep mask.

Embodiments of the present invention provide a sleep mask with blue light blocking lenses comprising a soft body member configured to fit comfortably over the orbital region of the face of a wearer; a pair of lenses configured to filter out blue light disposed in the soft body member; padding disposed perimetrically around an interior surface of the sleep mask in a continuous and uninterrupted manner; and a soft or pliant strap member disposed to secure the sleep mask upon the wearer's face, wherein the sleep mask conforms to the wearer's face as a soft surface against which the wearer is not discomforted when placing pressure against the sleep mask when worn; the pair of lenses consists of a first lens and a second lens, separate and distinct from the first lens, disposed in a non-overlapping, side-by-side arrangement; and the padding permits eyes of the wearer to open and close without contacting the sleep mask.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a rear diagonal elevation view of an example embodiment;

FIG. 2 is a front diagonal elevation view of an example embodiment;

FIG. 3 is a front elevation view of an example embodiment;

FIG. 4 is a rear elevation view of an example embodiment;

FIG. 5 is a ride side elevation view of an example embodiment;

FIG. 6 is a left side elevation view of an example embodiment;

FIG. 7 is a top elevation view of an example embodiment;

FIG. 8 is a bottom elevation view of an example embodiment;

FIG. 9 is a top elevation view of an example embodiment; and

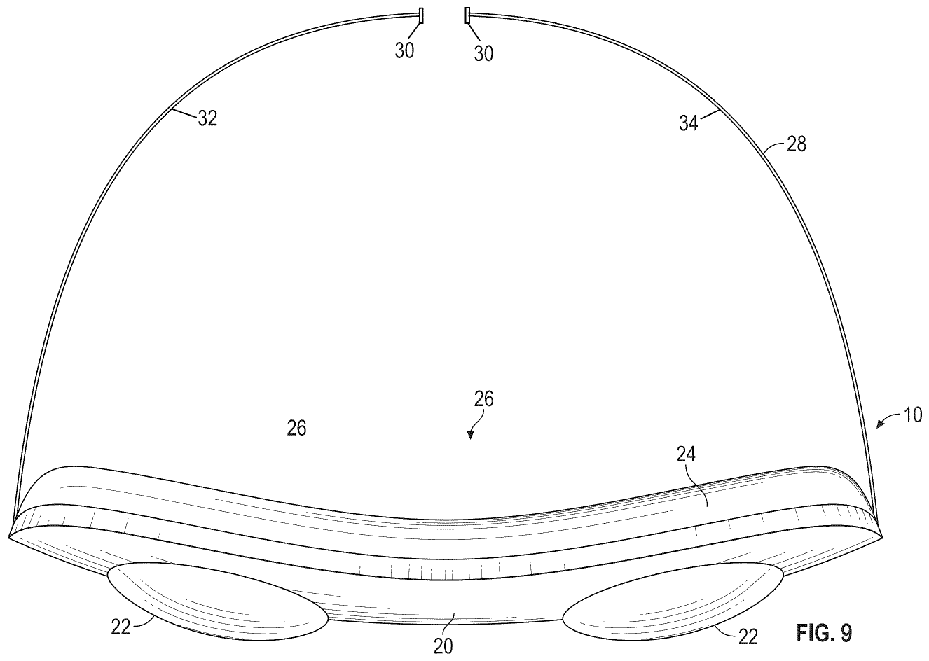

FIG. 10 is a front perspective view of an example embodiment.

The illustrations in the figures may not necessarily be drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a sleep mask with blue light blocking lenses that includes a soft body member having at least one lens configured to block blue light disposed therein. The mask is configured for comfort and secures over the orbital region of a wearer's face. The mask may include a padding to maintain the at least one lens away from contact with the wearer, even when a wearer lies face down wearing the mask. The at least one lens may be rendered of a flexible, non-rigid, translucent polymer. The wearer may therefore wear the mask while viewing screens in bed without exposure to blue light stimulating wakefulness.

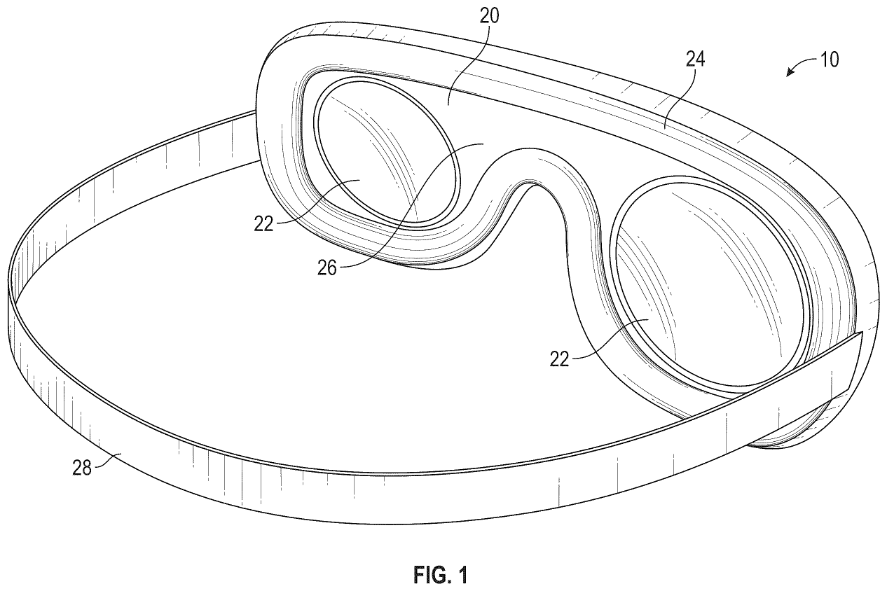

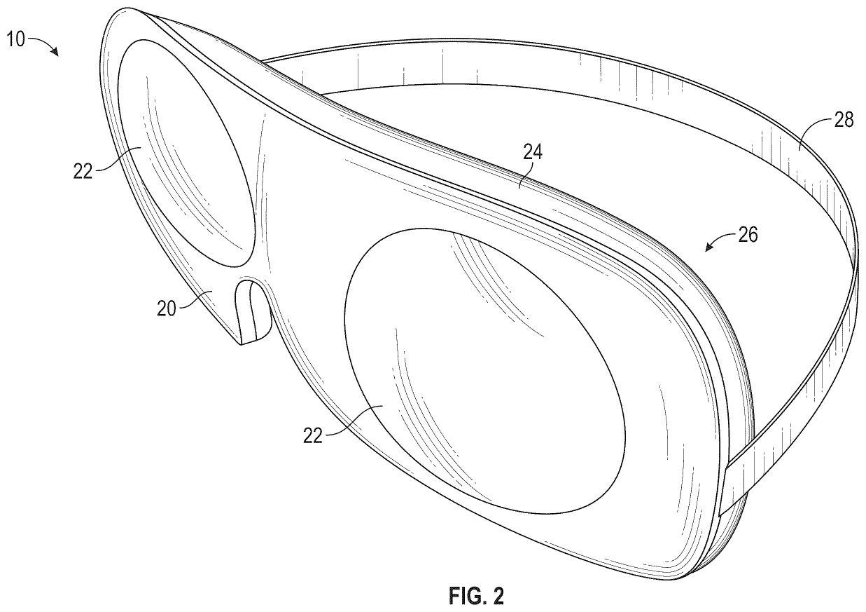

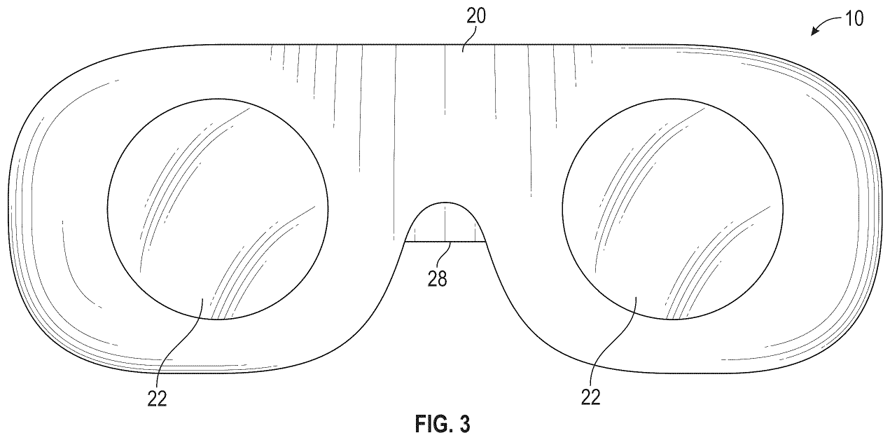

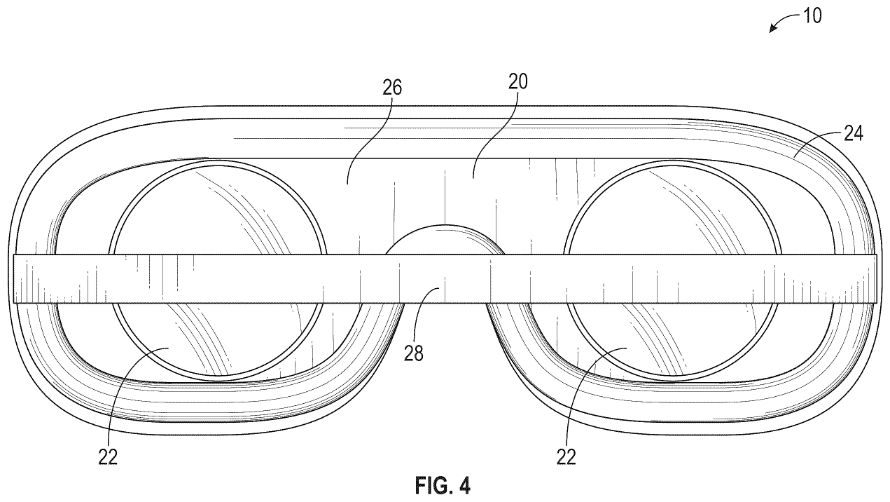

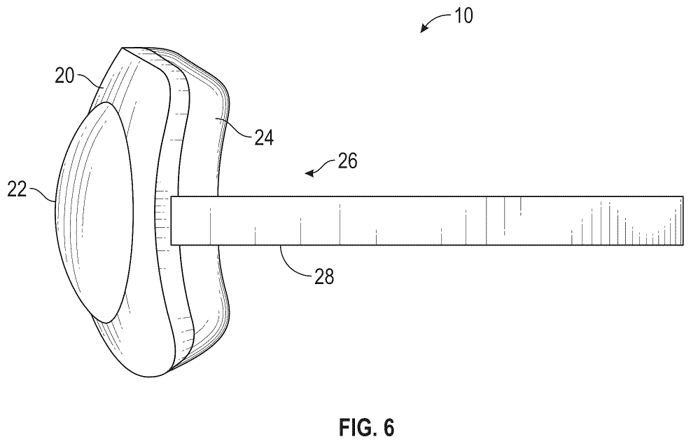

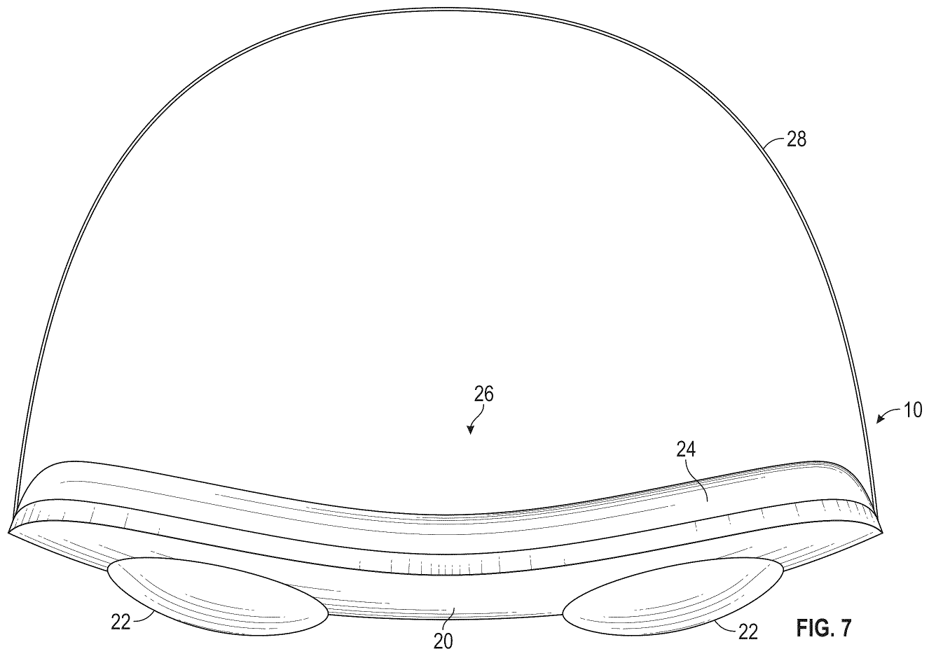

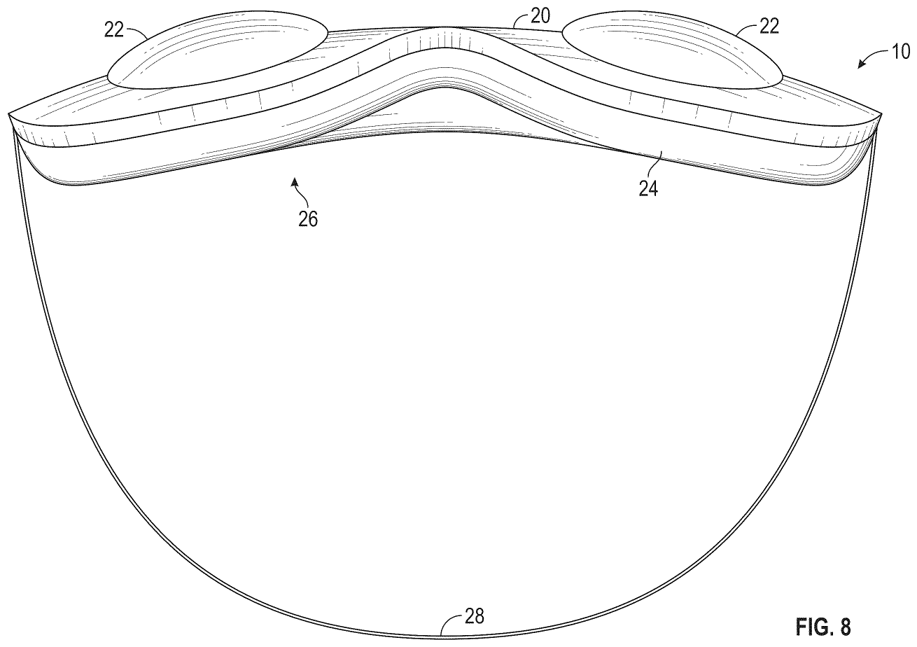

An example embodiment of the instant sleep mask with blue blocking lenses 10 is shown in FIGS. 1 through 8. Soft body member 20 is configured to comfortably fit upon the face of a wearer, over the orbital region around the wearer's eyes and the glabella and bridge of the nose. Soft body member 20 may be rendered of any soft or pliant or conformable material, including fabrics and polymers and other materials. Soft body member 20 includes pair of lenses 22. The pair of lenses 22 is recessed into body member 20 to prevent contact with a wearer's eyelashes when the sleep mask 10 is worn. The pair of lenses 22 is configured to filter out blue light and may comprise a flexible, translucent polymer, capable of bending or flexing at least to some degree to prevent rigidity of the mask 10 when worn. Alternatively, the pair of lenses 22 may be glass or other translucent material or polymer.

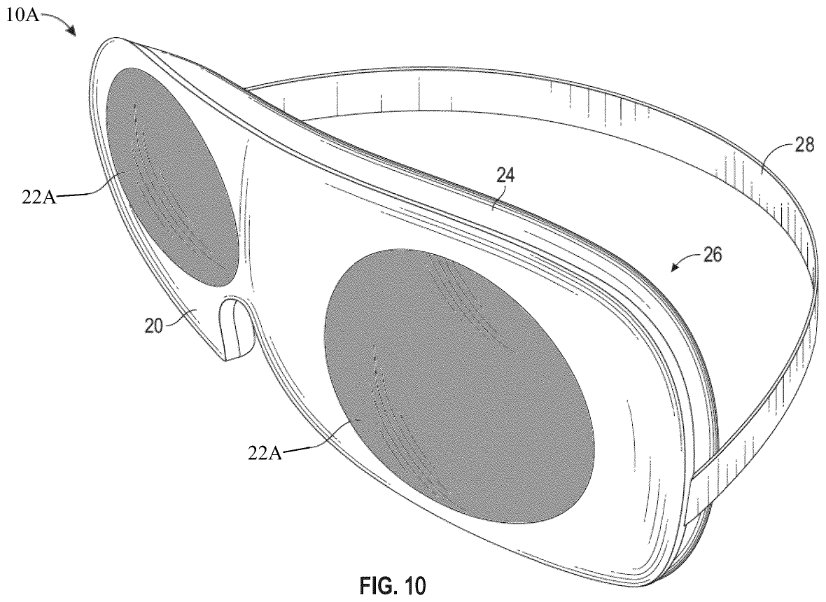

In some embodiments, as shown in FIG. 10, a pair of lenses 22A (also referred to as a pair of sunglass lenses 22A) may be polarized or formed to block sun rays or sun glare, similar to the lenses of sunglasses. Such soft-framed sunglasses can improve safety. For example, when wearing conventional sunglasses, if a user gets into a car accident, or other similar situation, the rigidity of the sunglasses may result in damage to the user's face, eyes, or the like. With the soft body member 20 and, in some embodiments, with flexibility in the pair of lenses 22A, injury can be minimized or prevented in the event of an incident that may result in impact to the user's face. In some embodiments, the pair of sunglass lenses 22A may also impart the features of the lenses 22, including being capable of blocking blue light.

In some embodiments, the pair of lenses 22 or the pair of sunglass lenses 22A may be formed as prescription/corrective lenses. Thus, the user can use the face mask even if the activity being performed requires the use of prescription/corrective lenses. The prescription/corrective lenses can include the feature of the lenses, as discussed above, including the ability to block blue light.

Soft body member 20 includes padding 24 disposed, in the example embodiment illustrated, perimetrically around interior surface of mask 10. The padding 24 can provide contact of the perimetric interior surface of the mask with the head of the user. Padding 24 may serve to maintain the mask 10 comfortably in position in contact with the wearer's forehead, orbital region around the eyes, the temples, and/or cheeks. Padding 24 ensures pair of lenses 22 is maintained spaced apart from contact with the wearer a sufficient distance to enable the wearer to open and close his or her eyes without contacting pair of lenses 22 or other parts of soft body member 20. Padding 24 may be compressible to ensure comfort when the wearer falls asleep with the mask on while maintaining sufficient capacity to keep the pair of lenses 22 from coming into contact with the wearer.

Alternative or other configurations of padding 24 are contemplated as within the instant application's disclosure, including padding 24 that does not surround the periphery of soft body member 20, but padding 24 incorporated at select positions upon interior surface 26 to maintain contact with a wearer while keeping pair of lenses 22 suitably removed from contact with the wearer. Such partial padding 24 may include padding disposed to contact the wearer at the glabella or interciliary space above the bridge of the nose, for example, or padding to contact key and select positions around the orbital region of the wearer. In some embodiments, an interior surface of sleep mask provides a user contact surface configured to contact a forehead of a user with a continuous and uninterrupted surface along an entirety of the user contact surface. This user contact surface can be defined by the padding 24, for example.

Mask 10 is secured to the wearer by soft or pliable strap member 28. Strap member 28 may be fastenable or elasticized or otherwise securable about the head of a wearer. Strap member 28 may be disposed interior to an external fabric sheath. Strap member 28 is soft and pliable to ensure comfort when worn and to remain comfortable when a wearer sleeps wearing the mask 10. Strap member 28 may be manufactured of elastic or inelastic material, and may include fastenable elements, or be disposed interior to a soft and comfortable sheath. Strap member 28 may comprise two, non-elastic members 32, 34 which are fastenable together. Strap member 28 may be rendered of fabric, polymer, or other soft and conformable material.

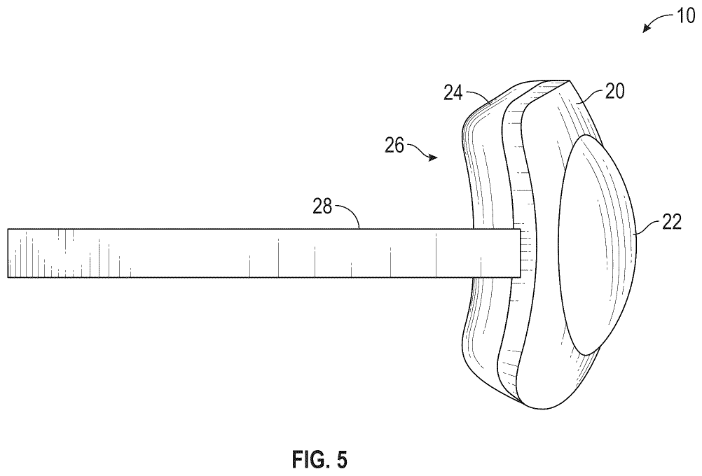

As is shown particularly in FIGS. 5 through 8, pair of lenses 22 may be shaped or mounted convexly in soft body member 20 to present a concavity on interior surface of body member 20 to thereby further prevent contact with the orbital region of the wearer.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A face mask with blue light blocking lenses comprising:
a soft body member configured to fit comfortably over the orbital region of the face of a wearer;
a padding about at least a portion of a perimetric interior surface of the soft body member;
a pair of lenses configured to filter out blue light disposed in the soft body member; and
a soft or pliant strap member disposed to secure the face mask upon the wearer's face, wherein:
the face mask conforms to the wearer's face as a soft surface against which the wearer is not discomforted when placing pressure against the face mask when worn; and
the padding permits eyes of the wearer to open and close without contacting the face mask.

2. The face mask with blue light blocking lenses of claim 1 wherein each of the pair of lenses is pliable and non-rigid.

3. The face mask with blue light blocking lenses of claim 2 wherein the strap member comprises two, non-elastic members which are fastenable together.

4. The face mask with blue light locking lenses of claim 2 wherein the face mask further comprises a padding disposed to contact at least the interciliary space of the wearer whereby the face mask softly fits covering the wearer's orbital region even when pressured against the wearer's face such as when the wearer is lying face down while wearing the face mask.

5. The face mask with blue light blocking lenses of claim 2 wherein the strap member is elastic.

6. The face mask with blue light blocking lenses of claim 5 wherein the strap member is disposed interior to a fabric sheath.

7. The face mask with blue light blocking lenses of claim 6 wherein the face mask further comprises a padding disposed to contact at least the interciliary space of the wearer whereby the face mask softly fits against the wearer's face even when pressured against the wearer's face such as when the wearer is lying face down while wearing the face mask.

8. The face mask with blue light blocking lenses of claim 6 wherein the face mask further comprises a padding disposed to at least selectively contact the wearer around the orbital region of the wearer's face.

9. The face mask of claim 1, further comprising padding disposed perimetrically around an interior surface of the face mask in a continuous and uninterrupted manner.

10. The face mask of claim 1, wherein the pair of lenses consists of a first lens and a second lens, at least one of which is a prescription lens.

11. The face mask of claim 1, wherein the pair of lenses consists of a first lens and a second lens, at least one of which is a sunglass lens.

12. A sleep mask with blue light blocking lenses comprising:
a soft body member configured to fit comfortably over the orbital region of the face of a wearer;
a pair of lenses configured to filter out blue light disposed in the soft body member;
a padding about at least a portion of a perimetric interior surface of the soft body member; and
a soft or pliant strap member disposed to secure the sleep mask upon the wearer's face, wherein:
the sleep mask conforms to the wearer's face as a soft surface against which the wearer is not discomforted when placing pressure against the sleep mask when worn;
the pair of lenses consist essentially of a first lens and a second lens disposed in a non-overlapping, side-by-side arrangement; and
the padding permits eyes of the wearer to open and close without contacting the sleep mask.

13. The sleep mask with blue light blocking lenses of claim 12 wherein each of the pair of lenses is pliable and non-rigid.

14. The sleep mask with blue light blocking lenses of claim 13 wherein the strap member comprises two, non-elastic members which are fastenable together.

15. The sleep mask with blue light locking lenses of claim 12 wherein the sleep mask further comprises a padding disposed to contact at least the interciliary space of the wearer whereby the sleep mask softly fits covering the wearer's orbital region even when pressured against the wearer's face such as when the wearer is lying face down while wearing the sleep mask.

16. The sleep mask with blue light blocking lenses of claim 13 wherein the strap member is elastic.

17. The sleep mask with blue light blocking lenses of claim 16 wherein the strap member is disposed interior to a fabric sheath.

18. A sleep mask with blue light blocking lenses comprising:
a soft body member configured to fit comfortably over the orbital region of the face of a wearer;
a pair of lenses configured to filter out blue light disposed in the soft body member;
padding disposed perimetrically around an interior surface of the sleep mask in a continuous and uninterrupted manner; and
a soft or pliant strap member disposed to secure the sleep mask upon the wearer's face, wherein:
the sleep mask conforms to the wearer's face as a soft surface against which the wearer is not discomforted when placing pressure against the sleep mask when worn;
the pair of lenses consists of a first lens and a second lens, separate and distinct from the first lens, disposed in a non-overlapping, side-by-side arrangement; and
the padding permits eyes of the wearer to open and close without contacting the sleep mask.

19. The sleep mask with blue light blocking lenses of claim 18 wherein each of the pair of lenses is pliable and non-rigid.

20. The sleep mask with blue light blocking lenses of claim 19 wherein the strap member comprises two, non-elastic members which are fastenable together.
